(19) United States
(12) Patent Application Publication
(10) Pub. No.: US 2007/0191693 A1
(43) Pub. Date: Aug. 16, 2007

(54) SYSTEMS AND METHODS FOR MEDICAL MONITORING AND HOME AUTOMATION

(76) Inventor: Robin Peng, Sandy, UT (US)

Correspondence Address: BAKER & ASSOCIATES PLLC, 470 EAST NINTH AVENUE, SALT LAKE CITY, UT, 84103, US

(21) Appl. No.: 11/670,979

(22) Filed: Feb. 3, 2007

Related U.S. Application Data
(60) Provisional application No. 60/765,107, filed on Feb. 4, 2006.

(51) Int. Cl. A61B 5/00; A61B 5/02; A61B 5/04

(52) U.S. Cl. 600/301; 600/549; 600/523; 600/509; 600/485; 128/920

(57) ABSTRACT

The present invention relates to systems and methods for monitoring and tracking attributes of personal health. One embodiment of the present invention relates to a method for measuring and recording at least two independent health values so as to produce multivariable diagnostic information that can accurately identify medical conditions. The method requires measuring at least two health related values which are recorded onto some form of computer readable media and then correlated concurrently to known medical conditions. These conditions are then communicated to the user An alternative embodiment relates to a system for performing multivariable health related diagnostics using at least two health related measurement devices, a computation processor, and an output device. The devices may be external devices configured to communicate with the remainder of the system. The output device may include a display screen to communicate visually and/or a speaker to communicate verbally.

### CROSS-REFERENCES TO RELATED APPLICATIONS

The present invention claims priority on earlier U.S. Provisional Application 60/765,107, filed Feb. 3, 2006, and incorporates said earlier Application herein by reference in its entirety.

### FIELD OF THE INVENTION

The invention generally relates to health care. In particular, the present invention relates to systems and methods for monitoring and tracking attributes of personal health.

### BACKGROUND OF THE INVENTION

Due to rising health care costs and increased public availability of medical information, individuals have begun to monitor their personal health independent of medical professionals. A large number of households currently include at least one health value measuring device such as a scale, blood pressure monitor, heart rate monitor, or body fat analyzer. These devices are used to measure at least one value related to an individual's overall health. For example, a scale is used to measure the body weight of a user. Body weight is often tracked to determine if weight is gained or lost over a particular time period. Likewise, weight may be used to self diagnose degrees of obesity which are known to lead to a variety of health related problems. In addition to elective health monitoring, many individuals are required to measure certain health values as a result of one or more medical conditions. For example, a diabetic must monitor his or her insulin levels to determine when it is necessary to ingest or inject supplemental insulin. Likewise, certain heart related conditions require that a patient regularly monitor his or her blood pressure.

In certain circumstances, medical professionals may be required to routinely measure one or more health values to ensure that a patient is healthy. Often, patients that require consistent measurements must be located in a hospital or medical care facility to allow medical professionals to analyze the values. For example, in certain individuals, rapid increases or decreases in blood pressure may indicate a severe medical condition, which must be treated immediately. If a patient is disabled mentally or physically it may not be practical to accurately and reliably perform these measurements. To address this problem, various companies have designed devices and systems to allow for the remote monitoring of patients. These systems allow participants to measure their values and report them to medical professional via some form of communication device. This enables the medical professional to remotely review the values to determine if further intervention is necessary.

However, the existing systems fail to overcome many of the problems encountered in pursuit of accurate, convenient medical value measurement. Participants and patients are often still required to manually measure one or more values. Participants who are mentally or physically incapacitated will not be able to utilize this type of system because of an inability to operate the measurement equipment. In addition, participants may then be required to record the measured values and transmit information to the doctor. These tasks may also be problematic for certain disabled individuals.

In addition, conventional remote health monitoring systems are directed exclusively for severely unhealthy individuals. These systems are designed exclusively to transmit information to a medical professional for review. Therefore, there is no local interpretation of the measured health values. The only information provided to the patient is the individual measurements of each particular device.

Likewise, conventional systems are prohibitively expensive. Each of the measuring devices is often packaged independently of the overall system, thereby requiring independent power sources, user interfaces, and transmission systems. Thus, a user may be required to operate two or more measurement devices in order to provide the necessary health values for proper assessment. These devices are packaged in a manner that prevents or impedes their disposal in a particular tight location, often requiring independent storage. Further, conventional monitoring systems require an operator to install the communication link between the patient and the medical monitoring professional.

Therefore, there is a need in the industry for a medical monitoring system that may be effectively utilized by both healthy and unhealthy individuals, minimizes physical space, minimizes overall cost, and is capable of reliably recording and monitoring health care value information.

### SUMMARY OF THE INVENTION

The present invention relates to systems and methods for monitoring and tracking attributes of personal health. One embodiment of the present invention relates to a method for measuring and recording at least two independent health values so as to produce multivariable diagnostic information. Multivariable diagnostic information can accurately identify conditions in which the independent health values may still be within normal ranges. The method requires measuring at least two health related values including but not limited to age, height, weight, sex, blood pressure, pain, etc. The health related values are recorded onto some form of computer readable media such as a hard disk. The health related values are then correlated concurrently to known medical and health related conditions. Correlated medical conditions are then communicated to the user via some form of output device. An alternative embodiment of the present invention relates to a system for performing multivariable health related diagnostics. The system includes at least two health related measurement devices, a processor, and an output device. The health related measurement devices may be external devices configured to communicate with the remainder of the system. The processor may be any form of computation processor. The output device may include a display screen to communicate visually and/or a speaker to communicate verbally.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

The following description of the invention can be understood in light of the Figures, which illustrate specific aspects of the invention and are a part of the specification. Together with the following description, the Figures demonstrate and explain the principles of the invention. The Figures presented in conjunction with this description are views of only particular—rather than complete—portions of the systems and methods of making and using the port system according to the invention. In the Figures, the physical dimensions may be exaggerated for clarity. The same reference numerals in different drawings represent the same element, and thus their descriptions will be omitted.

FIG. 1 illustrates a perspective view of one embodiment of a customized product in accordance with embodiments of the present invention;

FIG. 2 illustrates a flow chart of a method for measuring and recording at least two independent health values so as to produce multivariable diagnostic information in accordance with one embodiment of the present invention; and

FIG. 3 is a schematic illustrating a system for performing multivariable health related diagnostics in accordance with one embodiment of the present invention.

### DETAILED DESCRIPTION OF THE INVENTION

With reference now to the drawings, the preferred embodiment of the application tool is herein described. It should be noted that the articles “a”, “an” and “the”, as used in this specification, include plural referents unless the content clearly dictates otherwise.

The present invention relates to systems and methods for monitoring and tracking attributes of personal health. One embodiment of the present invention relates to a method for measuring and recording at least two independent health values so as to produce multivariable diagnostic information. Multivariable diagnostic information can accurately identify conditions in which the independent health values may still be within normal ranges. The method requires measuring at least two health related values including but not limited to age, height, weight, sex, blood pressure, pain, etc. The health related values are recorded onto some form of computer readable media such as a hard disk. The health related values are then correlated concurrently to known medical and health related conditions. Correlated medical conditions are then communicated to the user via some form of output device. An alternative embodiment of the present invention relates to a system for performing multivariable health related diagnostics. The system includes at least two health related measurement devices, a processor, and an output device. The health related measurement devices may be external devices configured to communicate with the remainder of the system. The processor may be any form of computation processor. The output device may include a display screen to communicate visually and/or a speaker to communicate verbally.

The following terms are defined as follows:

Multivariable diagnostic information—diagnostic information that is obtained by analyzing health related values concurrently rather than independently. Multivariable diagnostics may detect medical conditions from independent health related values even if the independent health related values appear normal when analyzed independently. Multivariable diagnostics require at least two measured health related values.

Health related values—Any type of information related to an individual's health including numerical values, images, sounds, samples, etc. Health related information includes but is not limited to height, weight, blood pressure, heart rate, insulin level, blood analysis, X-ray images, verbal cough, hair sample, etc.

Health related devices—A device that is in communication with a health care monitoring system. Health related devices may measure health related data values and transmit information to the health care monitoring system. In addition, a health related device may provide a health related function such as an emergency response button. Other health related devices include but are not limited to a weight scale, height measurement, a thermometer, a digital stethoscope, a blood pressure cuff, a pulse oximeter, an ECG/rhythm strip, a glucose meter, and a first aid kit.

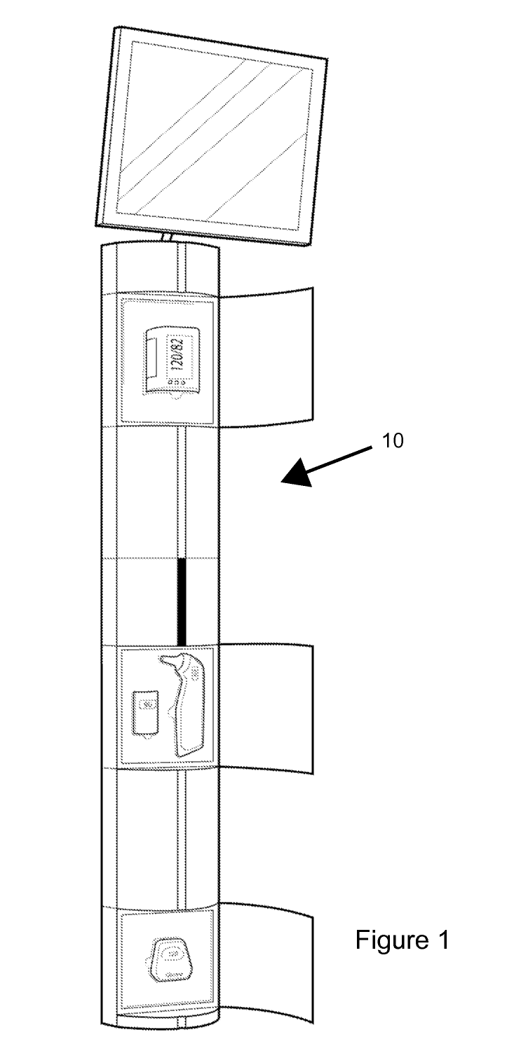

Reference is initially made to FIG. 1, which illustrates a perspective view of a product 10 produced in accordance with one embodiment of the present invention.

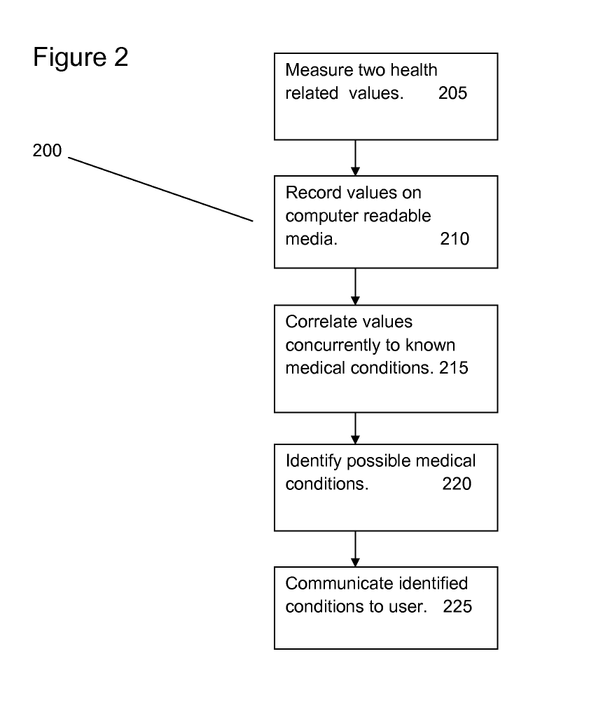

Reference is next made to FIG. 2, which illustrates a flow chart of a method for measuring and recording at least two independent health values so as to produce multivariable diagnostic information in accordance with one embodiment of the present invention, designated generally at 200. The method includes measuring at least two health related values, act 205. The act of measuring the at least two health related values may further include engaging and/or activating one or more external measurement devices. The at least two health related values are recorded onto a computer readable medium, act 210. The at least two health related values are correlated concurrently against known medical conditions, act 215, to identify possible medical conditions, act 220. Identified correlated medical conditions are communicated to a user via an output device, act 225.

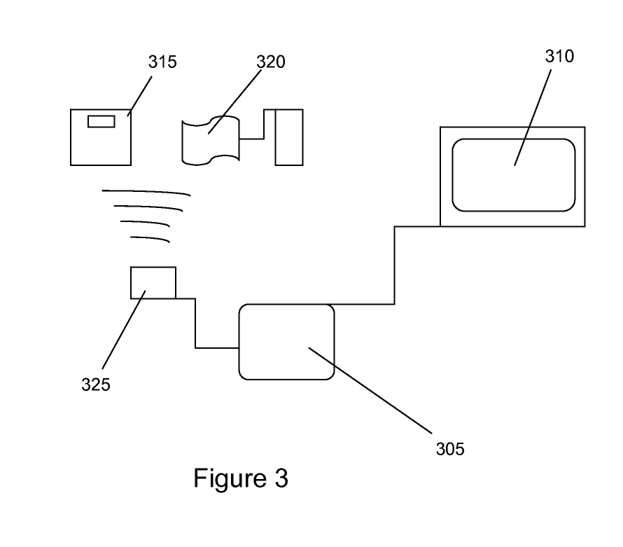

Reference is next made to FIG. 3, which illustrates a system for performing multivariable health related diagnostics in accordance with one embodiment of the present invention, designated generally at 300. The system 300 includes at least two health related measurement devices 315, 320. The illustrated health related measurement devices are a weight scale 315 and a blood pressure cuff 320 respectively. The system further includes a processor 305 configured to record and concurrently correlate health related measurements with medical data related to medical conditions. In the illustrated embodiment, the processor 305 is coupled to a communication module 325 which provides for wireless communication between the processor and the at least two measurement devices. The system further includes an output device 310. The illustrated output device is display screen coupled to the processor to display and communicate information to a user. It should be noted that any medical device may be used and any output or display device may be used while still practicing this invention.

Although the present invention has been described with reference to preferred embodiments, numerous modifications and variations can be made and still the result will come within the scope of the invention. No limitation with respect to the specific embodiments disclosed herein is intended or should be inferred.

What is claimed is:

1. A method for measuring and recording at least two independent health values so as to produce multivariable diagnostic information comprising the acts of:
a. measuring at least two health related values;
b. recording the at least two health related values onto a computer readable medium;
c. correlating the at least two health related values concurrently against known medical conditions; and
d. communicating correlated medical conditions via an output device.

2. The method of claim 1, wherein the act of measuring the at least two health related values includes engaging at least two independent health measuring devices which are in communication with a central processing unit.

3. A system for performing multivariable health related diagnostics comprising:
a. at least two health related measurement devices;
b. a processor configured to record and concurrently correlate health related measurements with medical data related to medical conditions; and
c. an output device configured to communicate correlated medical conditions to a user.

4. The system of claim 3, wherein the at least two health related measurement devices include a scale, height measurement device, a thermometer, a digital stethoscope, a blood pressure cuff, a pulse oximeter, a ECG/rhythm strip, a glucometer, and a first aid kit.
